SEQUENCE LISTING (4 sequences)

SEQ ID NO: 1 - RNA, 81 nt, synthetic construct
  source 1..81: mol_type = other RNA
tgtcaggcctgctagtcagccacagtttggggaaagctgtgcagcctgtaacccccccaggagaagctgggaaaccaagct

SEQ ID NO: 2 - RNA, 489 nt, synthetic construct
  source 1..489: mol_type = other RNA
tgtcaggcctgctagtcagccacagtttggggaaagctgtgcagcctgtaacccccccaggagaagctgggaaaccaagctcaacaacaacaaatgcgcacccttagcgagaggtttatcattaaggtcaacctctggatgttgtttcggcatcctgcattgaatctgagttactgtctgatttcctgaagtcgtgctgcttcatgtggtcggggtagcggctgaagcactgcacgccgtacgtcagggtggtcacgagggtgggccagggcaccggcagcttgccgaggaaacccgtttcttctgacgtaagggtgcgcattgacgcggttctatctagttacgcgttaaaccaactagaaagttcactgccgtataggcaggctgaagcactgcacgccatgtttaagagctatgctggaaacagcatagcaagtttaaataaggctagtccgttatcaacttgaaaaagtggcaccgagtcggtgc

SEQ ID NO: 3 - protein, 4 aa, synthetic construct
YADD

SEQ ID NO: 4 - protein, 4 aa, synthetic construct
YANN

(19) United States
(12) Patent Application Publication
(10) Pub. No.: US 2025/0236891 A1
(43) Pub. Date: Jul. 24, 2025

(54) OPTIMIZED RETRON EDITORS FOR EFFICIENT GENOME ENGINEERING AND DELIVERY TO MAMMALIAN CELLS

(71) Applicants: New York University, New York, NY (US); The Rockefeller University, New York, NY (US)

(72) Inventors: John Poirier, Brooklyn, NY (US); Matthew Cattle, New York, NY (US); Charles M. Rice, New York, NY (US); Lauren C. Aguado, New York, NY (US); William M. Schneider, New York, NY (US)

(21) Appl. No.: 19/030,612

(22) Filed: Jan. 17, 2025

Related U.S. Application Data
(60) Provisional application No. 63/623,240, filed on Jan. 20, 2024.

(51) Int. Cl. C12N 15/90; C12N 9/22; C12N 15/11

(52) U.S. Cl. CPC C12N 15/907; C12N 9/22; C12N 15/11; C12N 2310/20; C12N 2310/531; C12N 2800/80

(57) ABSTRACT

Provided are compositions, methods and system that relate to improved retron mediated modification of double stranded DNA. The compositions and methods include RNA polynucleotides that have a 5′ RNA structure that confers resistance to an RNA nuclease, an RNA sequence configured to be reverse transcribed into a single stranded DNA polynucleotide, a single guide RNA sequence targeted to a DNA editing site in a double stranded DNA template, a reverse transcriptase, and a nuclease that can nick at least one strand of the DNA editing site. Use of the systems overcomes low steady-state RNA levels that have hampered editing by previous retron-based systems in mammalian cells.

### CROSS-REFERENCE TO RELATED APPLICATIONS

This application claims the benefit of priority to U.S. provisional patent application No. 63/623,240, filed Jan. 20, 2024, the entire disclosure of which is incorporated herein by reference.

### SEQUENCE LISTING

The instant application contains a sequence listing which has been submitted in.xml format and is hereby incorporated by reference in its entirety. Said.xml file is named “058636.00786_Retron.xml”, was created on Jan. 17, 2025, and is 4,888 bytes in size.

### RELATED INFORMATION

Germline and somatic genetic variants play an important role in human health and disease. CRISPR-Cas9 technology has revolutionized the manipulation of genetic information by enabling RNA-programmable delivery of arbitrary enzymatic activities to targeted locations in the genome, facilitating new genome editing technologies that can interrogate the relationship between genotype and phenotype in cellular or organismal model systems. When delivered via lentivirus, linked genotype: phenotype relationships can be maintained and the fate of individually edited cells and their progeny tracked, significantly simplifying the scalable parallelization of forward genetic screens.

Of the approaches developed to introduce single nucleotide variants (SNVs), it is considered that only base editing and prime editing systems have been adapted to lentiviral delivery. Base editors can instill single-nucleotide variants with high efficiency but require distinct and highly engineered editing enzymes to generate different types of mutations, limiting their application in large-scale screens. In contrast, prime editors offer a greater potential to efficiently introduce a wide spectrum of mutation types, including small insertions; however, each prime editing guide RNA requires extensive design effort to yield efficient editing, complicating the construction of large pegRNA libraries. There remains a need for additional methods to complement current genome editing tools that have the properties of broad editing potential and predictable activity, preferably in a manner that are compatible with lentiviral delivery.

Retrons are a class of prokaryotic retroelements that have been adapted into genome editing tools by taking advantage of their ability to generate multicopy single-stranded DNA (msDNA) homology-directed repair templates in situ. Each retron minimally consists of a non-coding RNA (ncRNA) that is reverse transcribed into msDNA by its cognate primer-independent reverse transcriptase. Fusion of protein and RNA components of retrons to their respective CRISPR-Cas9 counterparts results in targeted, templated DNA repair. However, the application of retrons as genome editing tools in mammalian cells has been limited due to relatively low efficiency that results in a lack of activity in the context of lentiviral vectors and editing outcomes biased toward small insertions and deletions resulting from non-homologous end joining of Cas9-induced double strand breaks. Thus, there is an ongoing and unmet need for improved retron constructs and methods of using them. The present disclosure is related to this need.

### BRIEF SUMMARY

Using a model genome editing assay and northern blotting techniques, the present disclosure reveals a key determinant for poor retron editing efficiency in mammalian cells. The disclosure provides re-engineered retron non-coding RNAs that enable editing efficiency from a viral vector, demonstrated using a lentiviral vector integrated at low copy. The disclosure demonstrates low steady-state RNA levels as a key limiting factor for editing efficiency and demonstrates the use of RNA pseudoknot structures to improve steady-state RNA levels, which increases genome editing efficiency as further described below and in shown the accompanying figures. In examples, improvements on the described design are provided and include but are not limited use of a ribonuclease RNA processing strategy to improve nuclease editing activity and overall genome editing rates. These approaches enable genome editing efficiencies enriched in templated editing outcomes from a representative low-copy integrated locus delivered by lentivirus.

### BRIEF DESCRIPTION OF THE FIGURES

FIG. 1. Comparison of Eco1 retron to ssODN templated repair efficiency in a model genome editing assay. (Panel A) Schematic of Cas9-RT, msr-msd, and sgBFP expression vectors. (Panel B) Experimental setup for transfection-based retron editing. HEK293T cells were transfected with Cas9-RT and ncRNA expressing plasmids and assessed for editing outcomes by flow cytometry 72 h post-transfection. (Panel C) Representative flow cytometry plots showing gates used to capture editing outcomes. Gates were drawn using an untransfected control. (Panel D) Percentage of GFP positive cells by retron template source. In vitro transcribed RNA and GFP ssODN were transfected at two different concentrations, either 0.610 pmols or 6.10 pmols. Error bars denote standard error of the mean.

FIG. 2. Genome editing efficiency of ncRNA architectures and delivery strategies. (Panel A) HDR editing outcomes from transfection-based editing assay as indicated by percentage of GFP+ cells. msr-msd-sgBFP (v2) represents an editor ncRNA with proposed improved structures. (Panel B) Experimental setup for integrated ncRNA retron editing. Cells were transduced with ncRNA expressing lentivirus vectors and selected with puromycin 24 h later. After one week of outgrowth, cells were transfected with plasmids expressing Cas9-RT and the corresponding ncRNA component in the case of integrated msr-msd or sgBFP. (Panel C, Panel D) NHEJ and HDR editing outcomes from an integrated ncRNA-based editing assay as indicated by percentage of BFP+ or GFP+ cells. Components that were expressed from a transfected plasmid are indicated (tfx), while all other ncRNA components were integrated into the genome by lentivirus. Error bars denote standard error of the mean.

FIG. 3. Analysis of ncRNA integrity and quantity when expressed from plasmid and lentiviral vectors. (Panel A) Schematic of both 3′ and 5′sgBFP ncRNA orientations showing location of the radiolabeled probe in the scaffold sequence. (Panel B) Northern blot of retron editor ncRNA levels expressed from transfected plasmids for sgBFP, msr-msd-sgBFP, and sgBFP-msr-msd Radiolabeled probe targeted the scaffold sequence of the sgRNA. (Panel C) Northern blot showing RNA levels of sgBFP or sgBFP-msr-msd when expressed from either transient transfection of a plasmid or low copy integration of a lentivirus vector.

FIG. 4. Evaluation of strategies to rescue ncRNA expression. (Panel A) Diagrams of retron editor ncRNA designs incorporating stabilizing elements. Top: circRNA design using Twister ribozyme sequences and tRNA stem forming ligation sequences. Middle: polyA RNA design incorporating a polyA AATAAA sequence before the RNAP III termination sequence. Bottom: ncRNA designs with a protective pseudoknot on the 5′ end before the retron template. (Panel B) Northern blots of retron editor ncRNAs incorporating stabilizing elements. Radiolabeled probe targeted the scaffold sequence of the sgRNA. (Panel C) Proposed mechanism of xrRNA-mediated RNA stability. Unprotected editor ncRNAs are susceptible to nuclease-driven decay, while an xrRNA pseudoknot on the 5′ end of the ncRNA blocks degradation by cellular nucleases. (Panel D) GFP editing outcomes for ncRNA constructs incorporating stability-enhancing elements. Error bars denote standard error of the mean.

FIG. 5. Optimization of post-transcriptional processing of ncRNAs to restore a native sgRNA 5′ end. (Panel A) BFP cutting efficiency of low-copy integrated structural ncRNAs. (Panel B) Diagram of processed retron editor ncRNA. Either tRNA or Csy4 cleavage sequence allows separation of the retron template from sgRNA after transcription. (Panel C) GFP editing efficiency of transfected processed retron editor ncRNAs. dC55G: mutated proline tRNA for improved processing, tRNAPro: full proline tRNA sequence, Csy4-H29A: nuclease deficient Csy4 mutant. (Panel D) Diagram of pseudoknot protected and Csy4-protected retron editor ncRNAs. xrRNA-csy4 contains a single xrRNA pseudoknot on the 5′ end of the retron template, while xrRNA-evopreQ1-csy4 contains two pseudoknots that flank the retron template for both 5′ and 3′ protection after Csy4 processing. (Panel E) Editing of BFP (top) and GFP (bottom) before and after ncRNA optimization in low-copy integrated HEK293T cells. (Panel F) Same as Panel E of FIG. 5; performed in low-copy integrated K562 cells. Error bars denote standard error of the mean.

FIG. 6. (Panel A) Editing of blue fluorescent protein (BFP) (top) and green fluorescent protein (GFP) (bottom) in HEK293T cells by transfecting the indicated constructs. (Panel B) Same as panel A of FIG. 6 by electroporating K562 cells. Error bars denote standard error of the mean.

### DETAILED DESCRIPTION

Unless defined otherwise herein, all technical and scientific terms used in this disclosure have the same meaning as commonly understood by one of ordinary skill in the art to which this disclosure pertains.

Abbreviations and other terms used in this disclosure include the following:
BFP—Blue Fluorescent Protein.
Cas9—CRISPR-associated protein 9, an endonuclease used in genome editing.
CRISPR—Clustered Regularly Interspaced Short Palindromic Repeats.
circRNA—Circular RNA, a covalently closed RNA molecule resistant to exonuclease degradation.
Csy4—A CRISPR-associated endoribonuclease that cleaves RNA at specific recognition sites. In the context of a ncRNA, ‘csy4’ indicates a Csy4-cleavable RNA substrate.
Ec86 retron—A prokaryotic retroelement adapted for genome editing. Also known as Eco1.
evopreQ1—A pseudoknot RNA structure used for RNA stability enhancement.
GFP—Green Fluorescent Protein.
HDR—Homology-Directed Repair, a DNA repair mechanism using a homologous template.
lentiviral vector—A viral delivery system for introducing genetic material into cells.
mCherry—A red fluorescent protein used as a transfection marker in genetic experiments.
mpknot—A pseudoknot RNA structure designed to enhance stability and prevent degradation.
msDNA—Multicopy single-stranded DNA produced by retrons.
msdRNA—RNA from which msDNA branches via 2′,5′-phosphodiester linkage
msd—region of genome encoding msDNA
msr—region of genome encoding msdRNA
msr-msd—A retron construct combining msr and msd.
ncRNA—Non-coding RNA, which is not translated into a protein
NHEJ—Non-Homologous End Joining, a DNA repair mechanism without a template
pegRNA—Prime editing guide RNA, used in prime editing.
polyA—A polyadenylated tail added to RNA to enhance stability and translation.
pseudoknot—A tertiary RNA structure enhancing stability and inhibiting RNA degradation.
retrons—Prokaryotic retroelements, with modified versions being described herein.
reverse transcriptase—An enzyme that synthesizes DNA from an RNA template.
RNA—Ribonucleic acid, a molecule essential for coding, decoding, regulation, and expression of genes.
scaffold—The structural RNA sequence in sgRNA that binds CRISPR proteins like Cas9.
sgRNA—Single-guide RNA, directing the CRISPR machinery to target DNA sequences.
spacer—A sequence in sgRNA that determines the DNA target for genome editing.
ssODN—Single-stranded Oligodeoxynucleotide, used as a repair template in genome editing.
T2A—A ribosomal skipping peptide enabling simultaneous expression of multiple proteins.
U6 promoter—A strong RNA polymerase III promoter used for expressing short RNAs like sgRNAs.
xrRNA—A specific pseudoknot RNA structure protecting retron editor ncRNAs from degradation.

Every numerical range given throughout this specification includes its upper and lower values, as well as every narrower numerical range that falls within it, as if such narrower numerical ranges were all expressly written herein.

The disclosure includes all polynucleotide and amino acid sequences described herein directly or by reference. Each RNA sequence includes its DNA equivalent, and each DNA sequence includes its RNA equivalent. Complementary and anti-parallel polynucleotide sequences are included. Sequences from 80.00%-99.99% identical to any sequence (amino acids and nucleotide sequences) of this disclosure are included.

The disclosure includes all polynucleotide and all amino acid sequences that are identified herein by way of a database entry. Such sequences are incorporated herein as they exist in the database on the filing date of this application or patent. The disclosure includes a proviso that a described construct includes differences as compared to the naturally occurring Ec86 retron, also known as Eco1.

The disclosure includes RNA structures and modified retron structures, as described in the text and illustrated by the figures. Those skilled in the art will recognize the described structures, which include but are not necessarily limited to pseudoknots. In embodiments, the described retron constructs may comprise one, or more than one RNA structure, such structures including pseudoknots, and stem-loop structures that are not included in a pseudoknot. In examples, more than one RNA structure can be present at an end of a described RNA polynucleotide. In examples, an RNA structure used in a described RNA polynucleotide is an xrRNA structure. xrRNA structures are described in Vicens Q, Kieft J S. Shared properties and singularities of exoribonuclease-resistant RNAs in viruses. Comput Struct Biotechnol J. 2021 Jul. 26; 19:4373-4380. doi: 10.1016/j.csbj.2021.07.024. PMID: 34471487; PMCID: PMC8374639, from which the description of xrRNA structures is incorporated herein by reference. In examples, an RNA structure used with embodiments of the disclosure are approximately 60-120 nucleotides in length.

In embodiments the disclosure provides compositions, methods, and systems for improved retron mediating DNA editing. In an example, the disclosure provides a system for use in retron mediated double stranded DNA editing. A representative system comprises i) an RNA polynucleotide comprising a 5′ RNA structure that confers resistance to an RNA nuclease and an RNA sequence configured to be reverse transcribed into a single stranded DNA polynucleotide for use in the retron mediated DNA editing, said RNA polynucleotide optionally further comprising a 3′ structure that also confers resistance to an RNA nuclease; ii) a single guide RNA (sgRNA) sequence targeted to a DNA editing site in a double stranded DNA template; iii) a reverse transcriptase; and iv) a nuclease that can nick at least one strand of the DNA editing site. In an example, the system may use a ribonuclease such as Cy4 to process a described RNA polynucleotide that is a component of the system. In an example, a described 5′ RNA structure comprises an RNA pseudoknot structure. In an example, the system can include a sgRNA in the form of a segment that also comprises other described components, or the sgRNA may be provided in trans. In examples, a described RNA polynucleotide of the disclosure comprises a cleavable site that when cleaved releases the sgRNA from the polynucleotide, and wherein optionally cleavage of the sgRNA sequence promotes formation of a 3′ structure on the RNA polynucleotide that lacks the sgRNA sequence after the cleavage. In examples, the nuclease that can nick at least one strand of the DNA editing site of the DNA editing site may nick both strands, or only one strand. Suitable nucleases are known in the art and include but are not necessarily limited to a Cas9 nuclease. In examples, the Cas9 nuclease may be a Cas9 from Streptococcus pyogenes. When used as a single-strand only nickase, the SpCas9 nickases may include the known D10A mutation which inactivates its RuvC domain, resulting in cleavage of only the target strand. Likewise, the SpCas9 nickases may include the known H840A mutation in the HNH domain to result in a nick in only the non-target strand. The reverse transcriptase that is a component of a described system may be provided as a single, intact protein, or as components of a fusion protein, including but not necessarily limited to a fusion protein comprising a Cas9 segment and a reverse transcriptase segment. Suitable reverse transcriptases for use with the described system are known, and include but are not necssesarily limited to reverse transcriptases derived from Avian Myeloblastosis Virus (AMV), Moloney murine leukemia virus (M-MLV), HIV-1 reverse transcriptase, Marathon reverse transcriptase, and Gsl-IIC reverse transcriptase. In examples, a described RNA polynucleotide may be encoded by an expression vector, including but not necessarily limited to a lentivirus vector. As such, a described RNA polynucleotide may be transcribed from an integrated provirus in cells that in which double stranded DNA is edited as described herein. In examples, when using lentiviral vectors or other integrated polynucleotides, the cells that are modified by a described retron system have only one, or not more than 2, 3, or 4 copies of the integrated sequence and as such are considered to have a low copy number of integrations.

In examples, some or all of a described system may be introduced into cells by way of a a ribonucleoprotein (RNP) that comprises a nuclease, reverse transcriptase, or a combination of the nuclease and the reverse transcriptase.

In an aspect, the disclosure provides a method comprising introducing a described system into eukaryotic cells such that the double stranded DNA editing site in the double stranded DNA template in the cells is edited by recombination of the reverse transcribed single stranded DNA polynucleotide at the location in the double stranded DNA template that is targeted by the sgRNA.

In examples, the disclosure demonstrates that recombination of the reverse transcribed single stranded DNA polynucleotide at the location in the double stranded DNA template that is targeted by the sgRNA is more efficient than editing the double stranded DNA template that is targeted by the sgRNA using an RNA polynucleotide that does not comprise the 5′ RNA structure but includes an RNA sequence that is reverse transcribed into a single stranded DNA polynucleotide.

The disclosure also includes eukaryotic cells comprising a described system.

The disclosure is illustrated using Cas9 nucleases, but other CRISPR proteins may also be used, including but not necessarily limited to Cas12. The CRISPR proteins may be catalytically active or catalytically dead or single-strand nicking mutant derivatives that inactivate a single catalytic site.

Alternatives to the described Csy4 endonuclease configuration can be used, such as a different riboendonuclease, or an RNA polynucleotide that can cleave RNA in a site-specific manner, i.e., a ribozyme. In examples, a Cas 12a can target the DNA and process the RNA. In embodiments, a nuclease of this disclosure is specific for a site in a described RNA polynucleotide that is not endogenously present in polynucleotides of the recipient cells.

In embodiments, any described component of a system of this disclosure may be provided in cis or in trans. Thus, all components of a system may be provided by a single polynucleotide. In embodiments, one or more components are provided by more than one molecule. In embodiments, components of a described system may be provided using more than one polynucleotide. In embodiments, a component of a described system may be separately integrated into the genome of a recipient cell, apart from other components of the system.

In this disclosure, it is revealed that retron ncRNAs, unprotected sgRNAs, and RNA fusions of these RNAs are unstable in mammalian cells, limiting templated editing efficiency and Cas9 endonuclease activity. Through iterative optimization, the disclosure provides in embodiments ncRNA architecture comprising the Ec86 (Eco1) retron ncRNA from E. coli encoding an embedded template and flanked by exoribonuclease-resistant pseudoknots, all fused to an sgRNA via a Csy4 endonuclease-cleavable stem loop. This construct liberates the sgRNA from the ncRNA transcript, protecting both products from exonuclease degradation, enhancing genome editing efficiency in the context of transient transfection and from an integrated provirus where prior designs are inactive, greatly broadening the utility of retrons as genome editing tools.

As described herein, in embodiments the disclosure provides a template segment that can be provided as a reverse transcribed single stranded DNA (but involvement of double stranded DNA is not necessarily excluded from the disclosure). In embodiments the template is included in a described construct. In embodiments, two pseudoknots flank the retron template followed by a nuclease recognition site for cleavage. In embodiments, the template is provided in trans (i.e., a separate molecule).

The sequence of the template is not particularly limited. In non-limiting embodiments, the template is no longer than 10-1,000 nucleotides, inclusive and including all ranges of numbers there between. In embodiments, the template is approximately 100 nucleotides. In embodiments the template is no longer than 100 nucleotides. In embodiments, systems of this disclosure include a template that may be considered to permit introduction of a DNA cargo for insertion into a eukaryotic chromosome or extrachromosomal element. The sequence of the DNA cargo is not particularly limited. In embodiments, the template may be devoid of any sequence that can be transcribed, and as such may be transcriptionally inert. Such sequences may be used, for example, to alter a regulatory sequence in a genome, to result in knockout or other modification of an endogenous gene, which can be used treatment of a genetic disease, study of gene effects, chromatin modeling, DNA binding protein analysis, methylation studies, and the like. In embodiments, the template comprises at least one open reading frame, which may be operably linked to a promoter that is included with the DNA insertion template, or the template is linked to an endogenous cell promoter once introduced. In non-limiting embodiments, the template comprises an open reading frame that encodes a peptide, e.g., a peptide that can be translated and which may be, for example, from several to 50 amino acids in length. In embodiments, a coding sequence provided by the template encodes a cellular localization signal, and the encoded peptide or polypeptide may be transported to any particular cellular compartment. In embodiments, a coding sequence provided by the template comprises at least one antigenic determinant, e.g., an epitope, or a binding partner, such as an antibody or antigen binding fragment of an antibody, or a chimeric antigen receptor (CAR). In embodiments, the template encodes a selectable or detectable marker. In embodiments, the detectable marker comprises a fluorescent protein, such as green fluorescent protein (GFP), enhanced GFP (eGFP), blue fluorescent protein (BFP), mCherry, and the like. In embodiments, the template comprises a sequence that is intended to disrupt or replace a gene or a segment of a gene. In embodiments, the template corrects a mutation in a cell, including but not necessarily limited to an insertion, deletion, a transition or transversion. The disclosure includes producing both knock in and knock out gene modifications in cells, and transgenic non-human animals that contain such cells. In embodiments, the template encodes a nuclear localization signal appended to a protein or peptide. In embodiments, the template includes a sequence that includes a ribosomal skipping sequence. Ribosomal skipping sequences are known in the art and include, in non-limiting embodiments, the ribosomal skipping peptides T2A, P2A, E2A, and F2A.

In embodiments, any described retron construct may comprise linking nucleotides between two designated components.

In embodiments, a system or any component thereof may be introduced into eukaryotic cells using, for example, one or more expression vectors, including but not limited to viral vectors such a lentivirus vectors, or by direct introduction of ribonucleoproteins (RNPs), or using plasmids. Any described component of the polynucleotides may be separately integrated into the genome of a recipient cell.

In embodiments, use of a described system exhibits greater DNA editing frequency in a population of cells relative to a similar system that uses unmodified retron RNA. In embodiments, detectable markers and/or selection elements can be used to determine editing frequency. In embodiments, editing frequency can be measured, for example, by a change in expression in a reporter gene. In embodiments, editing efficiency is determined by measuring the number of cells within a population that experience an editing event, as determined using any suitable approach, such as by reporter protein expression. In embodiments, the disclosure provides for increased editing, such as within a population of cells, relative to a control. The control can be any suitable control, such as a reference value, or any value using a control experiment with unmodified retron constructs, or retron constructs that have different modifications than a modified retron of this disclosure.

The following Examples are intended to illustrate but not limit the disclosure.

### Example 1

### Ec86 Retron-Mediated Genome Editing Efficiency is Limited in Mammalian Cell Lines

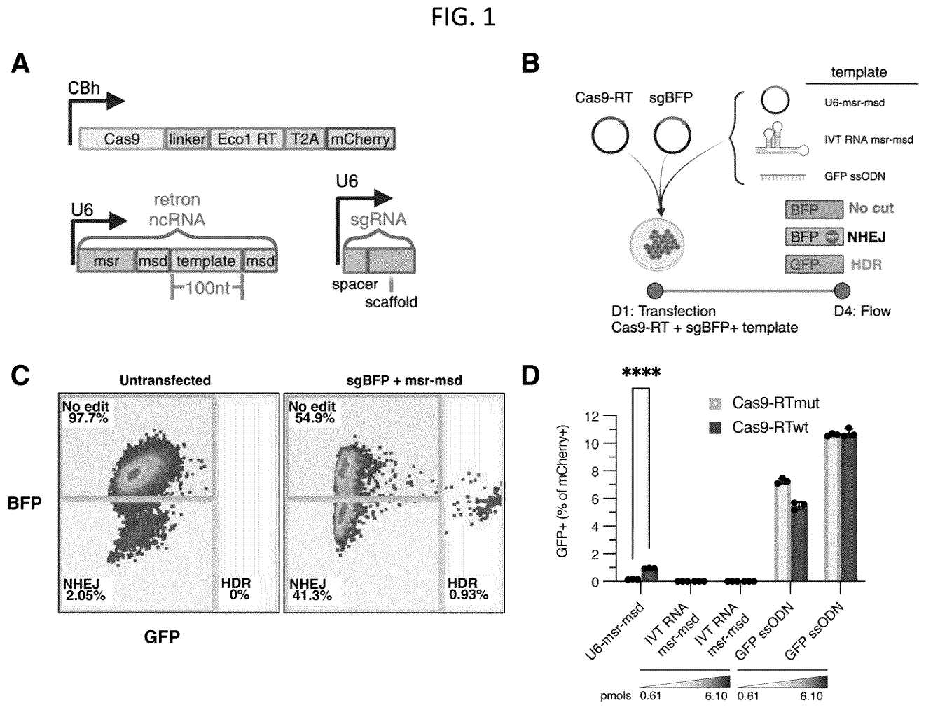

To analyze the cause of poor editing efficiency in mammalian cells, the disclosure uses modifications of the known Ec86 retron, which was optimized for mammalian expression, as a model retron for iterative enhancement of genome engineering. In an example, the protein-coding and non-coding components of the retron were expressed from independent plasmids. The protein-coding plasmid encodes a fusion protein comprising Cas9 fused to the Ec86 reverse transcriptase by a known XTEN amino acid linker followed by mCherry via the T2A ribosome skipping sequence to allow simultaneous tracking of transfected cells by red-orange fluorescence (FIG. Panel A, top). In order to control for background levels of plasmid templated repair, we generated an additional catalytically dead Ec86 mutant (YADD>YANN; SEQ ID NO:3 and 4, respectively) and confirmed the mutant is inactive by qPCR of the retron RT product. Non-coding RNA plasmids were designed to express the Ec86 ncRNA fused to an sgRNA and included a 100 nt repair template internal to the msd sequences. Retron editor ncRNAs were expressed from the U6 RNAPIII promoter and flanked by a polyU transcription termination signal, while constructs to make in vitro transcribed RNA were expressed from a T7 promoter and flanked by an HDV ribozyme to make precise 3′ ends. ncRNA vectors were designed to encode an sgRNA targeting the BFP chromophore (sgBFP) and a repair template encoding GFP and destroying the protospacer adjacent motif (PAM) of sgBFP (FIG. 1 Panel A, bottom).

To compare retron editor designs, we measured precision genome editing rates using an assay based on the conversion of an integrated blue fluorescent protein (BFP) to green fluorescent protein (GFP) by missense mutation of the chromophore. Using this system, the disclosure includes benchmarking retron editors delivered as plasmid DNA or as in vitro transcribed (IVT) ncRNA against a transfected ssODN repair template. We co-transfected the Cas9-RT expressing plasmid with either sgBFP alone, two independent plasmids expressing U6-sgBFP and U6-msr-msd with a 100 nt GFP repair template, U6-sgBFP with IVT msr-msd RNA containing GFP template, or U6-sgBFP with a GFP ssODN repair template (FIG. 1 Panel B). After 72 h we ran flow cytometry to quantify editing outcomes, drawing BFP− and GFP+ gates using an untransfected control (example outcome shown in FIG. 1 Panel C). While the plasmid-expressed ncRNA was able to edit the BFP locus to 0.95±0.01%, transfected RNA was unable to generate any successful recombination events. In contrast, an ssODN repair template of the same sequence as the plasmid-encoded msDNA GFP template greatly outperforms transfected retron editor ncRNA at inducing editing at the BFP locus, reaching 10.73±0.15% of transfected HEK293T cells recombining to GFP (FIG. 1 Panel D). These results are consistent with previous reports demonstrating poor editing efficiency in mammalian cells and indicate that ssDNA templates delivered via the Ec86 retron are comparatively poor substrates for editing versus ssODN.

### Example 2

### Retron Non-Coding RNA Abundance is Limiting in Mammalian Cells

A previously outstanding question with regard to retron editors is to what extent the context of ncRNA delivery and expression affects editing efficiency. Multiple factors related to how the nature of the delivered RNAs could reduce efficiency including lack of sgRNA tolerance to 5′ extension, disruption of secondary structures necessary for reverse transcription, and related complications. The disclosure provides analysis of several different aspects related to ncRNA delivery and architecture: cis versus trans expression of the retron ncRNA and sgRNA, different ncRNA orientations that alter the sgRNA position relative to the retron ncRNA, additional sequence structures proposed to improve RT efficiency and differences in relative expression of the retron template and sgRNA.

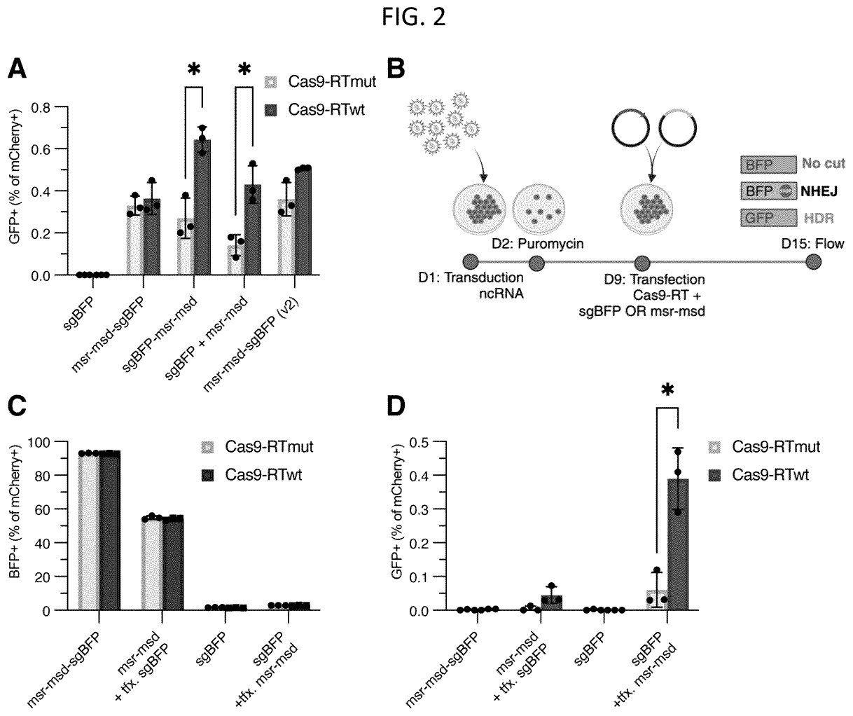

We initially compared editing efficiencies of cis versus trans expression of the sgRNA and retron template. For ncRNA delivery in cis, the disclosure also provides an analysis of how orientation of the sgRNA (5′ or 3′ relative to msr-msd template) and additional sequence features could improve RT affect retron editing efficiency. We combined several different sequence segments into a single ncRNA, including 14 nt extensions of the retron inverted repeats, 6 nt extension of the msd stem sequence, a 12 nt CAA-repeat linker between the retron msr-msd and sgRNA, and an alternative GFP repair template design of the non-coding GFP strand. We also compared these designs to a previously published retron editor plasmid, pBZ210, that expresses a 5′ sgBFP orientation retron editor ncRNA and the same Cas9-RT fusion protein from the same plasmid construct. In total, we compared five different retron editor ncRNA designs as well as sgBFP on its own. We observed that trans expression of the msr-msd and sgRNA and a 5′ sgRNA orientation had a significant increase in editing efficiency, while a 3′ sgRNA orientation saw no significant increase in GFP+ cells as compared to Cas9-RTmut regardless of additional ncRNA structures, indicating that the number of GFP+ cells overwhelmingly originates from plasmid-templated repair (FIG. 2 Panel A). Despite seeing improvements in retron-mediated editing when retron ncRNA and sgRNA are expressed in trans or with a 5′ sgRNA orientation, retron editing efficiency was limited even when nearly half of these successful were induced by plasmid-templated repair as seen by the percentage of GFP+ cells in the Cas9-RTmut conditions. The disclosure accordingly includes using the described configuration that improves results.

The importance of the relative abundance of the sgRNA and retron RNA template is facet of retron editing that is analyzed herein. The ability for retron editor ncRNA components to be expressed and operate in trans is harnessed to investigate the relative contribution of each component to steady-state abundance of the ncRNA-sgRNA fusion. By integrating at low multiplicity one of the retron editor ncRNA components, either the sgRNA or msr-msd template, and transfecting the other component at high-copy numbers to induce overexpression relative to the low-copy integrand, we analyzed the relationship between ncRNA abundance and retron editing efficiency (FIG. 2 Panel B). We were surprised to see that when integrated and expressed with Cas9-RT, sgBFP alone manages to induce NHEJ outcomes at very high frequencies (˜60%), but a representative retron editor ncRNA was unable to disrupt BFP at a frequency higher than ˜10% (FIG. 2 Panel C). The ability to disrupt BFP was restored when sgBFP was expressed in trans either by transfection or integration. Even more surprising, high editing percentages were observed (>3%) when sgBFP was integrated to retain Cas9 activity and the retron template was overexpressed by transfection (FIG. 2 Panel D). This result suggested that the retron RNA template levels could be limiting in these cells and that retron editing efficiency might be improved by increased abundance of the ncRNA. These results also indicate that fusion of the sgRNA to the retron msr-msd inhibits Cas9 nuclease activity, potentially by interfering with efficient RNP assembly.

### Example 3

Steady-State Levels of ncRNA are Reduced in Mammalian Cells

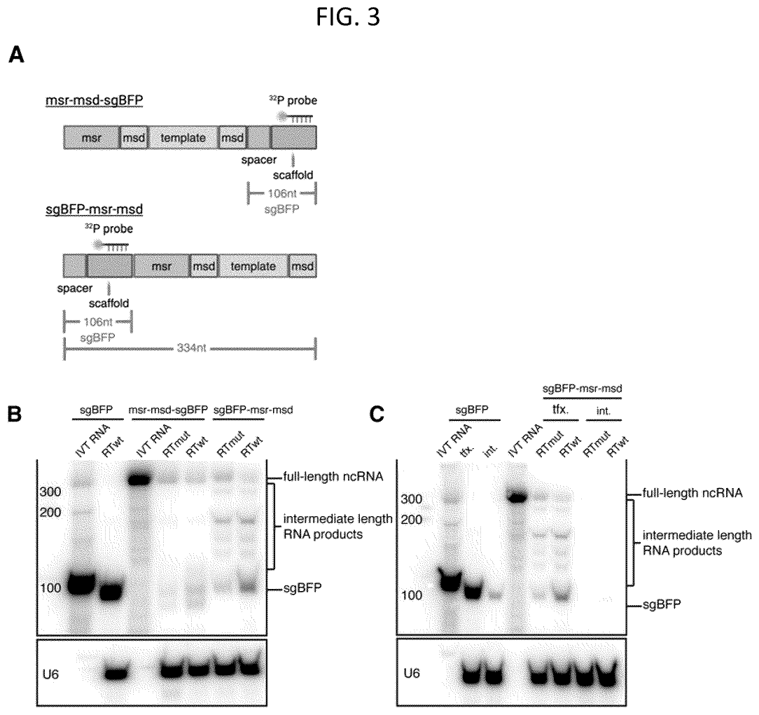

To detect steady-state levels of transfected retron editor ncRNAs, we designed P32 radiolabeled ssDNA probes to detect the sgBFP scaffold sequence of either a 106 nt sgBFP transcript or the 334 nt full-length sgBFP and msr-msd chimeric transcript by northern blot (FIG. 3 Panel A). Northern blot experiments revealed that co-delivery of Cas9 was required to stabilize sgRNAs. Therefore, we co-transfected HEK293T cells with Cas9-RT plasmids and either U6-sgBFP alone or 3′ and 5′ sgBFP retron ncRNAs and extracted whole-cell RNA to detect by northern blot. ncRNA abundance was significantly reduced as compared to expressing sgBFP without a fused retron ncRNA (FIG. 3 Panel B). We also observed that while the U6 snRNA loading control showed no evidence of RNA degradation, there was a consistent pattern of intermediate-length ncRNA products consistent with selective degradation by endogenous nucleases.

Given that editing efficiency from a low-copy lentivirus integration demonstrated inferior editing efficiency compared to transfection, we analyzed whether steady-state ncRNA levels would be further reduced in this context. We probed for ncRNA expression in a low-copy lentivirus integration cell line with a 5′ sgBFP ncRNA construct and compared expressed RNA to a cell line with low-copy integrations of sgBFP alone. We detected a band corresponding to full-length sgBFP when expressed from a transfected plasmid and from an integrated locus; however, in contrast we were unable to detect the full-length 5′ chimeric editor RNA when integrated despite the 5′ retron editor ncRNA being expressed from the same promoter as sgBFP alone (FIG. 3 Panel C). Lack of detectable band in both transfection and integrated blots along with specific banding pattern indicated potential RNA degradation.

### Example 4

xrRNA Knot Rescues Retron Editor ncRNA Steady-State Levels

The disclosure includes the following non-limiting demonstrations that correspond to the indicated figure panels.

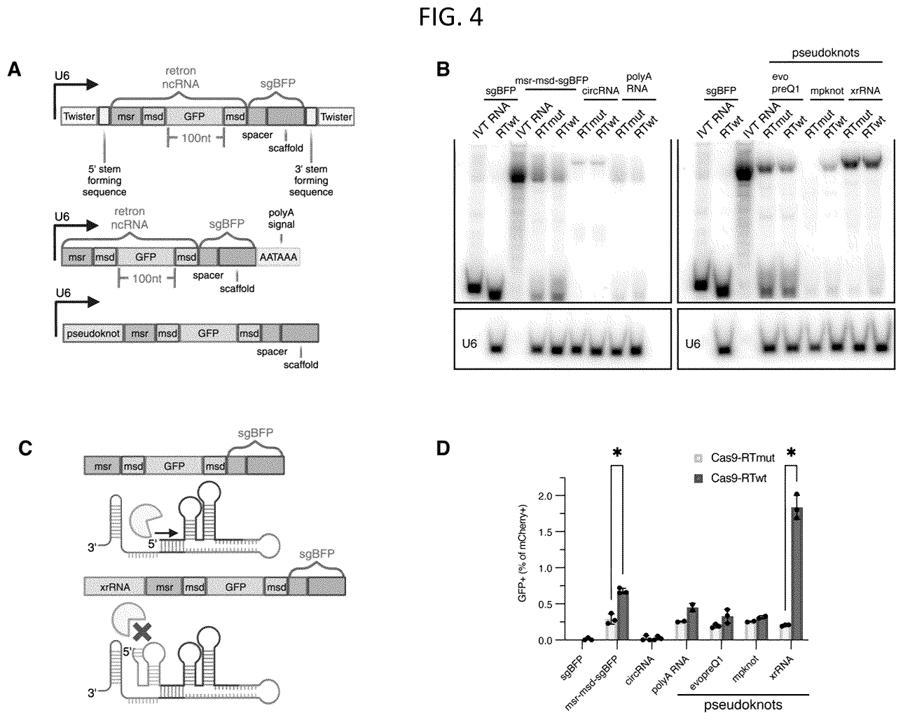

(FIG. 4 Panel A) Because free 5′ and 3′ RNA ends are frequently a substrate for ribonucleases and RNA sensing pathways, we analyzed whether hiding or otherwise protecting free RNA ends in a retron editor ncRNA could potentially provide stability and improve RNA abundance. In an effort to protect the retron editor ncRNA from mechanisms of RNA degradation, we adopted three strategies of RNA protection: RNA circularization, 3′ polyadenylation, and RNA tertiary-structure pseudoknots.

Circular RNAs (circRNA) are a type of covalently circularized RNA molecule that are found ubiquitously throughout nature. An aspect of circRNAs is that they are typically more stable than linear RNA due to their lack of exposed 5′ and 3′ phosphate ends, preventing exonuclease degradation. By taking advantage of tRNA ligation sequences and the endogenous tRNA ligase RtcB involved in tRNA processing, a circular gRNA conformation was expressed in cells and confirmed to increase half-life while reducing overall Cas9-gRNA DSB activity at both on and off-target sites.

The disclosure includes utilizing the advantage of the stability provided by polyA tails with RNAPIII-expressed retron editor ncRNAs by addition of an AATAAA polyA signal before the transcription termination signal.

We analyzed RNA tertiary structures as a method for RNA stabilization. We designed and cloned 5 unique ncRNA plasmids for circularizing, polyadenylating, and pseudoknot-protecting the retron editor ncRNA.

(FIG. 4 Panel B) Whole-cell RNA was extracted from HEK293T cells transfected with the different secondary structure-containing ncRNAs alongside either Cas9-RTwt or Cas9-RTmut and run on a northern blot. As seen in the northern blot, the xrRNA structure grafted to the 5′ end of the retron editor ncRNA most strongly rescued RNA abundance and reduced the total amount of intermediate-length products that were identified on the blot.

(FIG. 4 Panel C) Without intending to be bound by any particular theory, it is believed the depicted xrRNA structure provides stability to the described retron editor ncRNA by inhibiting RNA degradation.

(FIG. 4 Panel D) We then compared editing efficiencies of these different secondary structure-containing ncRNAs using the BFP to GFP gene editing assay. When transfected alongside Cas9-RTwt, the xrRNA-msr-msd-sgBFP reached GFP editing rates as high as ˜1.8%, ˜3-fold higher than a retron editor ncRNA that lacks any pseudoknot. None of the other ncRNAs were able to improve editing rates over a base ncRNA design that lacks any additional sequence, and the circular RNA design had significantly impaired Cas9 cutting activity and <0.1% editing efficiency. The improved editing efficiency of the xrRNA-containing ncRNA alongside increased RNA abundance as seen by blot indicates that RNA steady-state levels are perhaps a limiting factor for retron-mediated recombination. By improving the total amount of RNA through stability enhancing RNA structures demonstrated using pseudoknots, the disclosure provides an approach to address a major limiting factor for retron editing and greatly improved editing rates with a transfected retron template.

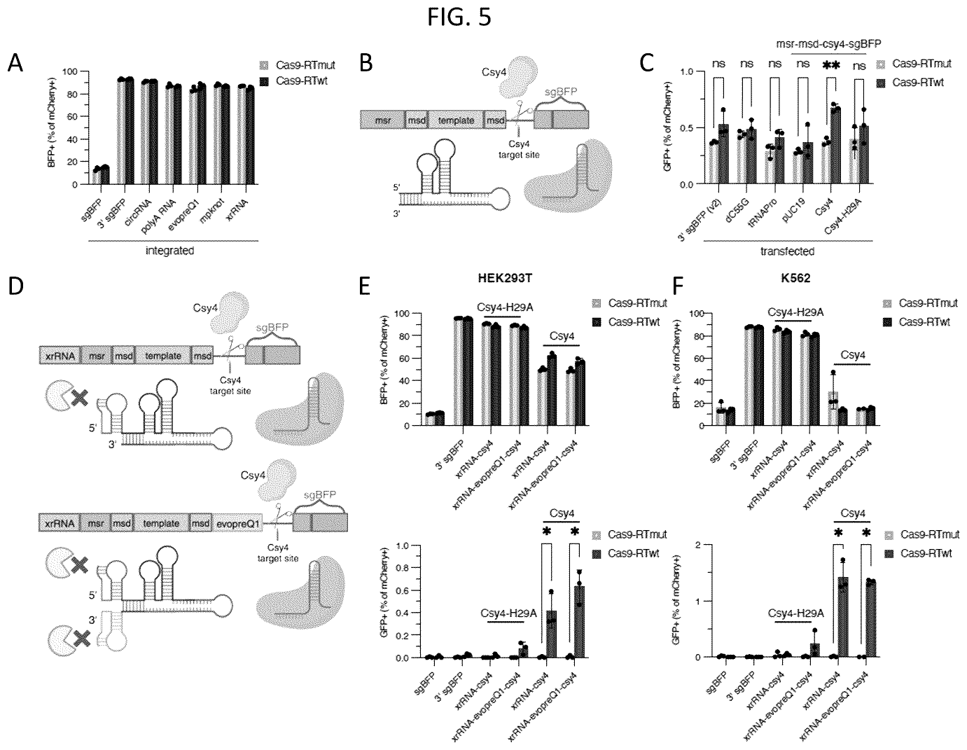

(FIG. 5 Panel A) We analyzed whether the improvements provided by the xrRNA pseudoknot in the transient transfection context would allow for genome editing when expressed at low multiplicity. Despite the improvements in RNA abundance and editing efficiency provided by the xrRNA-msr-msd-sgBFP transcript in the plasmid transfection context, this RNA design was only able to reach BFP inactivation of ˜14% with a Cas9-RTwt construct and as a result saw a complete lack of GFP editing. The lack of Cas9 activity in these cells despite containing a protective pseudoknot on the ncRNA indicates potentially another limitation of the ncRNA design besides low steady-state availability.

(FIG. 5 Panel B) We analyzed whether we could improve Cas9 cutting with a described retron editor by cleaving the msr-msd retron template from the sgRNA after expression, thereby liberating the sgRNA and creating a precise 5′ end.

In eukaryotes, tRNAs are naturally processed at their 5′ and 3′ ends by endogenous RNase P and RNase Z to create precise sequence ends. The disclosure includes adapting this RNA cleavage process by incorporating a proline tRNA sequence in between the retron RNA template and sgRNA. However, tRNAs also have internal RNAP III promoters. In order to maintain cleavage activity while reducing internal promoter activity, we also incorporated an engineered proline tRNA sequence (dC55G) that reduces internal promoter expression but is able to sustain RNA processing. This shorter engineered tRNA sequence also serves to reduce total transcript length. These described components are included in the scope of the disclosure.

Csy4 is a site-specific CRISPR endoribonuclease that processes sgRNA arrays by cleaving RNA at a 28 nt motif sequence. We cloned a 20 nt minimal Csy4 recognition site in between the retron template and sgBFP to make the ncRNA vector msr-msd-csy4-sgBFP. When expressed with the Csy4 endonuclease, this ncRNA should be cleaved to separate the retron RNA template from the sgRNA.

When transfected alongside Cas9-RTwt and a plasmid expressing wild-type Csy4, the Csy4-cleaved retron editor ncRNA improved both Cas9 cutting and retron-mediated recombination with the GFP repair template. This representative improvement is Csy4 cleavage dependent, as increased editing was not seen when transfected with a plasmid expressing a nuclease deficient Csy4 mutant, Csy4-H29A.

(FIG. 5 Panel C) In order to overcome the problems of low steady-state RNA and low sgRNA activity of chimeric ncRNA retron editor architectures, we combined both strategies of pseudoknot protection and Csy4 cleavage into a single ncRNA construct. We designed and cloned xrRNA-msr-msd-csy4-sgBFP, a ncRNA expressing lentiviral vector that has an xrRNA pseudoknot grafted to the 5′ end of the msr-msd template and a Csy4 recognition site located between the template and the sgRNA. To address exposing the 3′ end of the retron template to nucleases after cleavage we also used the second-best performing candidate pseudoknot seen in the northern blot and cloned an additional ncRNA vector, xrRNA-msr-msd-evopreQ1-csy4-sgBFP, that has two pseudoknots flanking the retron template followed by a Csy4 recognition site for cleavage.

(FIG. 5 Panel D) When tested in low-copy HEK293T cell lines, a double pseudoknot and Csy4-cleavable ncRNA led to a 9-fold increase in Cas9 cutting along with a 27-fold increase in editing rates when compared to the unoptimized retron editor ncRNA, up to a detectable mutation rate of 0.63±0.12%.

(FIG. 5 Panel E) We validated these improvements in the K562 chronic myelogenous leukemia cell line. When the xrRNA-msr-msd-evopreQ1-csy4-sgBFP ncRNA vector was expressed at low copy along with transiently transfected Cas9-RTwt and wild-type Csy4, we observed missense mutations rates of 2.9±0.07% versus <0.01% using the unoptimized ncRNA construct.

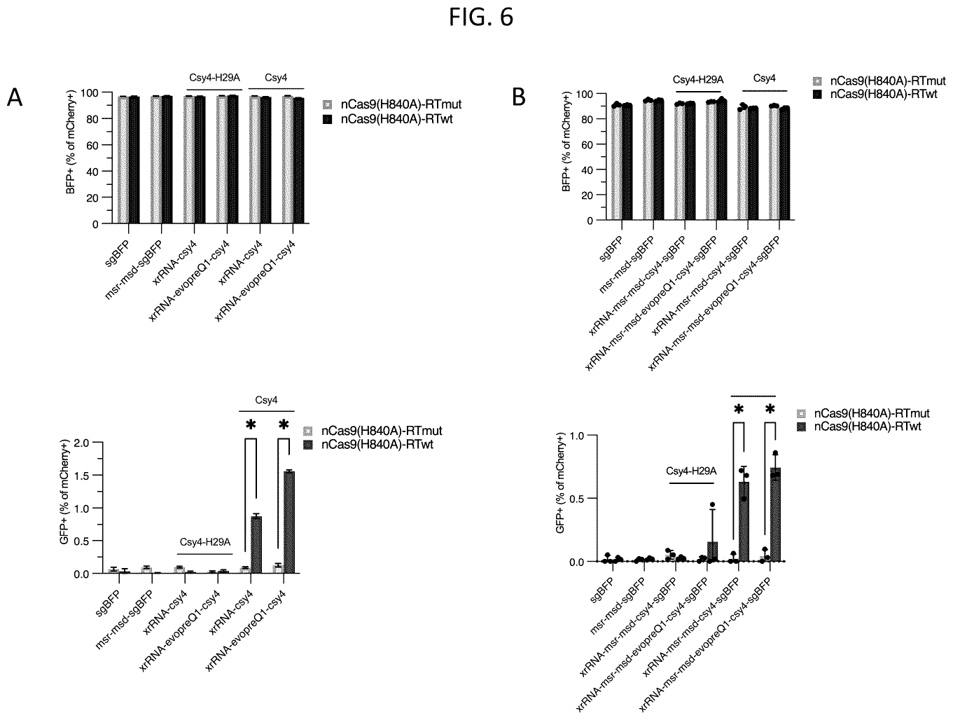

Given the significant enhancement in templated editing driven by both stability of the ncRNA and Cas9 cutting activity offered by Csy4 processing, we analyzed whether it would be possible to achieve templated editing using a Cas9 mutant that only nicks one strand of the target DNA, thereby reducing bias toward editing outcomes that result in insertions and deletions in favor of templated repair. We transfected HEK293T (FIG. 6 Panel A) and K562 (FIG. 6 Panel B) reporter cells with nCas9 (H840A)-RT, which cleaves only the non-target strand. These results clearly demonstrate reduced BFP disruption as well as the requirement for ncRNA protection and Csy4 processing for efficient templated repair. xrRNA-Csy4 is minimally required while xrRNA-evopreQ1-Csy4 is a preferred configuration. Unlike previous examples, insertions and deletions were minimized as demonstrated by minimal loss of BFP positivity.

### DISCUSSION

Using a model genome editing assay, this disclosure reveals the underlying basis for poor editing efficiency and extensively re-engineered the retron ncRNA to enable targeted rewriting of the genome from a lentiviral vector in two different mammalian cell lines. The disclosure demonstrates that low steady-state levels of the ncRNA as measured by northern blot significantly limits editing efficiency. To overcome this limitation, the disclosure includes analysis of a series of retron editor ncRNA variants that incorporate stability-enhancing secondary structures. Among the structures tested, a nuclease-resistant RNA pseudoknot from the Zika virus 3′ UTR rescued steady-state RNA levels most efficiently. Transient transfection of a plasmid expressing the pseudoknot-protected ncRNA resulted in a nearly 3-fold increase in templated repair.

The disclosure includes improvements on this design by using the Csy4 ribonuclease to process the sgRNA and retron ncRNA components. Delivering this pseudoknot-protected Csy4-cleavable ncRNA to HEK293T cells by low-copy lentivirus integration led to a 9-fold increase in Cas9 cutting along with a 27-fold increase in editing rates when compared to the unoptimized retron editor ncRNA, up to a detectable mutation rate of 0.63±0.12%. In the K562 chronic myelogenous leukemia cell line, the described improvements of the ncRNA enabled targeted missense mutation rates as high as 2.9±0.07% when expressed from an integrated locus versus <0.01% using the unoptimized ncRNA construct. Given that prior retron editor designs fail to induce genome editing in mammalian cells when delivered by lentivirus and are not able to produce templated edits without also causing double strand breaks, this improved design is considered to represent a significant advance in retron-mediated genome engineering technology. The data support use of the described polynucleotides, proteins and systems for massively parallel mutagenesis in mammalian cells, large-scale variant screening, somatic genome engineering, synthetic biology, and for prophylactic and therapeutic purposes in mammalian cells and mammals, including but not necessarily limited to human and non-human mammalian cells. Use in eukaryotic microorganisms and virally infected mammalian cells, to for example, target a viral genome or genome transcript, is included within the scope of the disclosure.

Representative materials and methods used to produce results described herein are as follows:
1.1. Plasmids and cell lines
1.2. Virus production
   T25 flasks were seeded with 2e6 HEK293T cells 24 h before transfection. On the following day, a transfection solution was made up of 200 μL OptiMEM, 3 μg of the ncRNA vector, 2 μg of psPax2, 1 μg of pMD2.g, and 12 μg of PEI. After mixing, the transfection solution was vortexed for 15 seconds, incubated at room temperature for 15 minutes, and finally added to cells drop wise. The media was replaced 24 h after transfection. On day 3 post-transfection, the media was harvested and filtered through a 0.45 μM syringe filter.
1.3. Transfections
   Lipofectamine 3000 was used for all transient transfections of HEK293T-BFP cells. Briefly, 2e5 HEK293T-BFP cells were reverse transfected with 750 ng of either Cas9-RTwt or Cas9-RTmut, and 650 ng of plasmids expressing the retron editor ncRNA. For all experiments incorporating Csy4 cleavage, 750 ng of either Csy4 wild-type or Csy4-H29A was added to the transfection mix, and conditions without a Csy4 recognition site were co-transfected with 750 ng pUC19 to balance total transfected mass. Cells were replated in a 12 well plate approximately 16-24 hours after transfection.
   The Neon NXT Electroporation System was used to electroporate K562-BFP ncRNA-expressing cell lines with Cas9-RT plasmids and one of Csy4, Csy4-H29A, or pUC19 to balance total transfected mass. 5e5 cells were pelleted at 300 RCF for 5 minutes and resuspended in Neon NxT R buffer and then mixed with 2500 ng of the Cas9-RT plasmid and 2500 ng of Csy4/Csy4-H29A/pUC19 plasmid. Cells were then electroporated using the following parameters in a 10 μL syringe: 1050V, 20 ms pulse width, 2 pulses. Cells were added to 500 μL of pre-warmed RPMI media in a 48 well plate after pulsing.

It will be recognized from the foregoing description that the present disclosure demonstrates the following non-limiting examples.

In one example, Zika xrRNA pseudoknot is shown to protect retron editor ncRNA. The disclosure includes improving retron editor efficiency in mammalian cells by rescuing full-length RNA steady-state levels. In examples, the Xrn1-resistant pseudoknot from the Zika virus 3′ UTR (xrRNA) showed a high degree of retron editor ncRNA rescue when expressed from plasmid. In the described model genome editing assay, retron editor ncRNAs with an xrRNA pseudoknot grafted to the 5′ end improve editing rates. Improvement of steady-state RNA levels and genome editing with an xrRNA pseudoknot implicate low RNA template availability as a limiting factor for retron editors.

The disclosure demonstrates that Csy4 ribonuclease cleavage liberates sgRNA from ncRNA. When xrRNA retron editor was expressed from a low multiplicity lentiviral integrand, the sgRNA failed to efficiently cut BFP despite showing high steady-state RNA levels. Failure to efficiently cut BFP suggests inhibited sgRNA by RNA fusion with retron RNA template. In order to improve sgRNA activity of retron editor ncRNAs, the disclosure includes use of RNA cleavage mechanisms to liberate the sgRNA from the retron RNA template. We attempted RNA processing by tRNA sequences (dC55G, tRNAPro) and Csy4. The disclosure shows that Csy4 cleavage and pseudoknot protection permit retron editing by lentivirus. In order to overcome RNA degradation and inhibited sgRNA, the disclosure includes a combination of pseudoknot end-protection strategy and the Csy4 RNA cleavage into a single ncRNA design. When expressed at low multiplicity via lentivirus integration, the Csy4-cleaved pseudoknot-protected retron editor ncRNA was able to induce genome editing in representative cell lines.

Demonstrations of the described system used the ZIKV xrRNA1 that comprises the following sequence:

(SEQ ID NO: 1)
UGUCAGGCCUGCUAGUCAGCCACAGUUUGGGGAAAGCUGUG
CAGCCUGUAACCCCCCCAGGAGAAGCUGGGAAACCAAGCU.

Non-limiting demonstrations of the described system used an RNA construct to produce results, including those shown in FIG. 5 Panel E, with a construct referred to as xrRNA-msr-msd-evopreQ1-csy4-sgBFP that comprised the following sequence:

(SEQ ID NO: 2))
ugucaggccugcuagucagccacaguuuggggaaagcugugcagc
cuguaacccccccaggagaagcugggaaaccaagcucaacaacaa
caaAUGCGCACCCUUAGCGAGAGGUUUAUCAUUAAGGUCAACCUC
UGGAUGUUGUUUCGGCAUCCUGCAUUGAAUCUGAGUUACUgucug
auuuccugaagucgugcugcuucauguggucgggguagcggcuga
agcacugcacgccguacgucaggguggucacgagggugggccagg
gcaccggcagcuugccgaggaaacccguuucuucugacguaaggg
ugcgca^{UUGACGCGGUUCUAUCUAGUUACGCGUUAAACCAACUAGAAA}
_{GUUCACUGCCGUAUAGGCAG}GCUGAAGCACUGCACGCCAUgu
uuaagagcuaugcuggaaacagcauagcaaguuuaaauaaggcua
guccguuaucaacuugaaaaaguggcaccgagucggugc

In the sequence shown above, the following components are included:
xrRNA shown in plain text
12 bp linker shown in lowercase italics
Msr shown in uppercase
msd (includes BFP to GFP repair template) shown in bold
evopreQ1 shown in superscript
Csy4 cut site shown in subscript
sgRNA spacer (targets BFP) shown in uppercase and italics
sgRNA scaffold—3′ 86 nucleotides

The described construct was used with transiently transfected Cas9-RTwt and wild-type Csy4.

The disclosure includes each annotated segment of SEQ ID NO:2 alone, and in all combinations, and DNA sequences encoding each annotated sequence, alone and in combination. SEQ ID NO:2 may be used with any desired segment but wherein the described msd segment used as a BFP to GFP repair template in SEQ ID NO:2 is replaced by any other desired repair template.

What is claimed is:

1. A system for use in retron mediated double stranded DNA editing, the system comprising:
i) an RNA polynucleotide comprising a 5′ RNA structure that confers resistance to an RNA nuclease and an RNA sequence configured to be reverse transcribed into a single stranded DNA polynucleotide for use in the retron mediated DNA editing, said RNA polynucleotide optionally further comprising a 3′ structure that also confers resistance to an RNA nuclease;
ii) a single guide RNA (sgRNA) sequence targeted to a DNA editing site in a double stranded DNA template;
iii) a reverse transcriptase; and
iv) a nuclease that can nick at least one strand of the DNA editing site.

2. The system of claim 1, wherein the 5′ RNA structure comprises an RNA pseudoknot structure.

3. The system of claim 1, wherein the RNA polynucleotide of i) comprises the sgRNA sequence of ii).

4. The system of claim 3, wherein the RNA polynucleotide comprises a cleavable site that when cleaved release the sgRNA from the polynucleotide, and wherein optionally cleavage of the sgRNA sequence promotes formation of the 3′ structure on the RNA polynucleotide that lacks the sgRNA sequence after the cleavage.

5. The system of claim 1, wherein nuclease is a Cas9 nuclease.

6. The system of claim 5, wherein the Cas9 nuclease is configured to only cleave one strand of the DNA editing site in the double stranded DNA template.

7. The system of claim 1, wherein the nuclease and the reverse transcriptase a components of a fusion protein.

8. The system of claim 1, wherein the RNA polynucleotide of i) is encoded by an expression vector and wherein the RNA polynucleotide is transcribed within cells after introduction of the expression into the cells.

9. The system of claim 1, wherein the polynucleotide of i) is present in a ribonucleoprotein (RNP) that comprises the nuclease, or the reverse transcriptase, or a combination of the nuclease and the reverse transcriptase.

10. A method comprising introducing a system of claim 1 into eukaryotic cells, and wherein the double stranded DNA editing site in the double stranded DNA template in the cells is edited by recombination of the reverse transcribed single stranded DNA polynucleotide at the location in the double stranded DNA template that is targeted by the sgRNA.

11. The method of claim 10, wherein the system is introduced into the eukaryotic cells using an expression vector from which the RNA polynucleotide of i) is transcribed.

12. The method of claim 10, wherein the system is introduced into the into the eukaryotic cells as a ribonucleoprotein (RNP) that comprises the RNA polynucleotide of i), and the nuclease, or the reverse transcriptase, or a combination of the nuclease and the reverse transcriptase,

13. The method of claim 10, wherein the 5′ RNA structure comprises an RNA pseudoknot structure.

14. The method of claim 13, wherein recombination of the reverse transcribed single stranded DNA polynucleotide at the location in the double stranded DNA template that is targeted by the sgRNA is more efficient than editing the double stranded DNA template that is targeted by the sgRNA using an RNA polynucleotide that does not comprise the 5′ RNA structure but includes an RNA sequence that is reverse transcribed into a single stranded DNA polynucleotide.

15. The method of 13, wherein the RNA polynucleotide comprises the sgRNA sequence.

16. The method of claim 15, wherein the RNA polynucleotide is cleaved to thereby release the sgRNA from the polynucleotide, and wherein cleavage of the sgRNA sequence promotes formation of the 3′ structure on the RNA polynucleotide that lacks the sgRNA sequence after the cleavage.

17. Eukaryotic cells comprising the system of claim 1.
